(19) United States
(12) Patent Application Publication
(10) Pub. No.: US 2026/0090933 A1
(43) Pub. Date: Apr. 2, 2026

(54) CATHETER MANAGEMENT DEVICE AND METHOD FOR CONTINENCE CARE

(71) Applicant: Virginia Commonwealth University, Richmond, VA (US)

(72) Inventors: Emma Shawcross, Powhatan, VA (US); Casey Grey, Richmond, VA (US)

(21) Appl. No.: 19/255,071

(22) Filed: Jun. 30, 2025

Related U.S. Application Data
(60) Provisional application No. 63/665,297, filed on Jun. 28, 2024.

(51) Int. Cl. A61G 7/07; A61M 27/00

(52) U.S. Cl. CPC A61G 7/07; A61M 27/00; A61M 2202/068; A61M 2209/084

(57) ABSTRACT

A positioning device with a wedge shape includes a channel on the underside configured to accommodate a fecal management catheter to prevent leaks and improve waste containment. The underside may include a first material for contact with the bed surface, and the channel may comprise a second material which is different from the first material and which is non-stick. The positioning device prevents back flow and leakage of contents onto patient/bed which can result if a catheter is blocked.

### CROSS-REFERENCE TO RELATED APPLICATIONS

This application claims the benefit of U.S. Provisional Patent App. No. 63/665,297, filed Jun. 28, 2024, the complete contents of which are herein incorporated by reference.

### FIELD OF THE INVENTION

Embodiments generally relate to fecal management and, more particularly, devices and methods which improve consistent and desired operation of fecal management catheters to improve patient outcomes.

### BACKGROUND

Fecal management systems (FMS) are widely used in critical care/inpatient settings for a variety of reasons. For instance, FIG. 1 shows Flexi-Seal™ PROTECT Fecal Management System 100 sold by Convatec® that includes an indwelling fecal management catheter 101 intended for use to manage fecal incontinence through the collection of liquid to semi-liquid stool and to provide access to administer medications. The end 102 of the catheter is affixed to the patient. The end 103 of the catheter is affixed to a receptacle such as bag 104. The bag 104 may include measurement indicia.

A problem with existing FMS systems such as the Flexi-Seal™ is they can fail to prevent back flow and leakage of contents onto patient/bed, a problem which arises when, for instance, the catheter is accidently crushed beneath the patient's weight or objects surrounding the patient.

FIG. 2 depicts a standard positioning wedge 201 in use beneath the knees 222 of legs 221 of a supine patient to provide elevation and stability to the patient. The need for positioning devices for patients often arises as a separate concern from continence care. Unfortunately, standard bed pillows/positioning wedges are not designed to accommodate-gravity flow of waste via fecal management devices. As a result, standard positioning devices do not assist with aforementioned problems of FMS systems and, in some cases, can amplify the problems. Traditionally, the bottom/underside 202 of the positioning wedge 201 is a flat continuous surface in contact with the bedding 203 beneath. Moreover, the positioning wedge 201 may span most of the bed width. The positioning wedge's shape and size make it a significant obstacle to routing catheters to/from the patient. The positioning wedge 201 can also shift position relative to its surroundings, such as when the patient moves, and wind up pinching, crushing, or kinking catheters.

### SUMMARY

Some exemplary embodiments are directed to fecal management systems/flow positioning systems which enhance fecal management by securely positioning patients and catheters to prevent leaks and improve waste containment.

Some exemplary embodiments improve the use and operation of existing FMS systems including but not limited to the Flexi-Seal™ PROTECT Fecal Management System and various waste management catheters.

According to some exemplary embodiments, a foam positioning wedge is provided which, according to a method of use, may be placed below a patient's knees to allow and ensure unimpeded flow within a fecal management system. The foam positioning wedge includes a channel on an undersurface of the wedge in which a catheter is fully accommodated and thus protected from pinching, crushing, kinking, etc. which could block flow within the catheter. The foam positioning wedge secures and protects the catheter while simultaneously maintaining alignment of the critically ill patient in bed (e.g., hob 30 degrees; hips aligned with indicator on bed rail, etc.).

Exemplary embodiments have the advantage of protecting waste management catheters from accidental limiting or blocking of flow. Further resultant advantages are prevention of back flow and leaking from an obstructed or blocked catheter. Leaks from waste management systems can have significant harmful consequences to patients such as skin breakdown from prolonged contact with waste and infection from contact of waste with open wounds. Exemplary embodiments protect patients from leaks and resultant health risks like skin breakdown and infection.

Some exemplary embodiments include positioning a patient around an exemplary device and preventing back flow and leakage of contents onto patient/bed which result if a catheter is blocked.

Some exemplary embodiments include accommodating gravity flow of waste via a fecal management device. Some exemplary devices include a channel configured to accommodate at least one waste (e.g., fecal waste) management catheter.

Exemplary applications of embodiments include, but are not limited to, inpatient setting adult intensive care unit (ICU), including liver patients, patients at risk for graft versus host (accuracy of output measurements is imperative), patients with skin breakdown, and wound healing issues due to diarrhea and debility.

For some patients and conditions, accuracy of output measurements from an FMS is important, even imperative. For instance, fecal waste output quantification for patients at risk for graft versus host is very important. Blocked fecal management catheters result in inaccurate waste output measurements. Accordingly, some exemplary embodiments of this disclosure have the advantage of improving or ensuring the accuracy of fecal output measurements for fecal matter collected with an FMS system such as but not limited to the Flexi-Seal™ PROTECT Fecal Management System (FIG. 1).

Some exemplary embodiments include an ergonomic design that ensures patient comfort and simplifies cleaning, promoting a more hygienic environment, improving wound healing and diminishing new wounds, and helps with precision management of stool output volume.

In some embodiments, an exemplary device is single-use. In some embodiments, an exemplary device is multiuse. In some embodiments, an exemplary device includes a washable cover. In some embodiments, an exemplary device includes a disposable cover and a reusable foam over which the cover is applied.

Exemplary channels of some exemplary devices may have one or more of the following features. In some embodiments, a channel may have a different surface than a remainder of the surfaces of the device. In some embodiments, the channel may have a nonstick surface, whereas other surfaces including top and/or bottom surfaces may have grippier surfaces. At least one surface of a channel may have a lower coefficient of friction than top and/or bottom surfaces of the device. A lower coefficient of friction as with a nonstick surface ensures a catheter, which may be made of silicone or plastic for example, can slide freely within the channel even if in contact with a channel wall.

An exemplary device comprises an underside, a topside, a frontside, a backside, and a channel. The backside is configured to face the foot of a bed on which a patient lies. The frontside is configured to face the head of the bed in such a state of use.

In some embodiments, an exemplary topside comprises one or more patient support surfaces. Such surfaces are configured to be load bearing, subject to external forces in a state of use. In particular, a patient support surface is configured to withstand forces acting on the patient support surface which result from a patient's weight acting along the vector of Earth's gravity.

Patient support surfaces may be directly contacted by a patient's body with no or little intervening materials. In general, some amount of fabric such as from a hospital gown or bedsheet may in some use cases be between a part of a patient and a patient support surface of a topside of exemplary device. The topside includes at least one surface which is directly above the channel.

In some embodiments, an exemplary underside comprises one or more underside surfaces which are configured to transfer loads acting on the device to an underlying environmental structure such as a bed or table. These surfaces of the underside are intended to make contact with the environment. These surfaces are to sides of the channel which is at the underside of the device.

Exemplary embodiments include one or more channels (at least one channel) running from one side to another side. An exemplary direction of the channel is between backside and frontside, though alternative embodiments may have a channel which runs between frontside and left side or frontside and right side of the device. In any case, the channel opens out at either end of at least two ends. The channel has a first opening (e.g., a backside opening) and a second opening (e.g., a frontside opening). The channel may have no floor. The channel may simply be open to the environment along its length in the downward direction. In use, when the device is placed on a bed or similar surface, the underlying surface such as the bedding creates a floor to the channel, and the channel becomes a tunnel.

When the device is subject to loading, as from some amount of patient weight being applied to a topside patient support surface, the channel does not collapse. The frontside opening, the backside opening, and empty space between the opposing openings remain intact. The device may be formed of a lightly deformable material such as certain medical grade foams, but even in such cases the material or materials are selected so that any deformation from patient loading of the device at the topside does not collapse the channel or its openings. Slight deformation of a channel may be permitted, but the minimum channel height even after accepted deformation is at least as great as the outer diameter of the catheter positioned in the channel.

An exemplary channel is sized to accommodate a catheter and, in particular, a fecal management catheter. The channel height is preferably at least as great as the outer diameter of a fecal management catheter with which the device is intended to be compatible. In some embodiments, a minimum channel height is at least 5% or at least 10% greater than the minimum fecal management catheter diameter. In any case, the channel is sized and shaped so that an entirety of the catheter may be situated inside the channel for the length of the channel. Because the channel is non-collapsible, the channel prevents external forces, especially those from above, from transferring to the catheter and risking deformation, crushing, or collapse of the catheter.

An exemplary channel is open at the underside of the device body. Such a channel may be referred to as a three-sided channel even if the channel has an inverted U-shape in cross-section or some other cross-sectional shape the number of sides of which may number greater or less than three from a strict geometric counting. In a state of use, the channel is closed on the fourth side by an underlying surface, generally the top of a hospital bed. For purposes of this disclosure, “bed” generally refers to a solid surface (often but not necessarily padded) which may have a covering of one or more linens and of shape and orientation so as to allow a patient to lie supine or substantially supine according to at least one configuration of the bed. Many hospital beds permit some degree of adjusting the angle of the bed relative to horizontal such that one or more top surfaces of the bed are adjustable in angle from an absolute horizontal position.

In some embodiments, an exemplary device comprises a body characterized by a monolithic structure, a single body. In some embodiments, an exemplary device comprises or consists of foam. An exemplary device has a body the predominant shape of which is a wedge. Alternatively, embodiments may have bodies in shapes other than a wedge.

### BRIEF DESCRIPTION OF THE DRAWINGS

FIG. 1 is a fecal management system (FMS) that includes a fecal management catheter and a collection receptacle.

FIG. 2 is a standard positioning wedge in a state of use providing elevation and stability to a patient's legs.

FIG. 3A is a perspective view of an exemplary positioning device.

FIG. 3B is an elevational view of the positioning device of FIG. 3A but inverted so that the device's underside is wholly visible.

FIG. 3C is an elevational view of the positioning device of FIG. 3A.

FIG. 4 is a picture of an exemplary positioning device in a state of use.

FIG. 5A is another exemplary positioning device which includes a wedge and a retainer which is insertable into a recess of the wedge.

FIG. 5B is the positioning device of FIG. 5A with the retainer fully inserted into the wedge.

FIG. 5C is a bottom plan view of the wedge from FIG. 5A.

FIG. 6A is a first exemplary insert for retaining a catheter in a channel.

FIG. 6B is a second exemplary insert for retaining a catheter in a channel.

FIG. 6C is a third exemplary insert for retaining a catheter in a channel.

FIG. 7 is another exemplary positioning device which includes a retainer which is integral with a wedge.

FIG. 8 is still another exemplary positioning device.

FIGS. 9A, 9B, and 9C are non-limiting exemplary alternative wedge profiles.

FIGS. 10A, 10B, and 10C are non-limiting exemplary alternative cross-sectioned wedges with different channel configurations.

FIGS. 11A, 11B, and 11C are non-limiting exemplary alternative channel configurations.

FIG. 12A is a further exemplary positioning device.

FIG. 12B is still another exemplary positioning device.

FIG. 12C is yet another exemplary positioning device.

### DETAILED DESCRIPTION

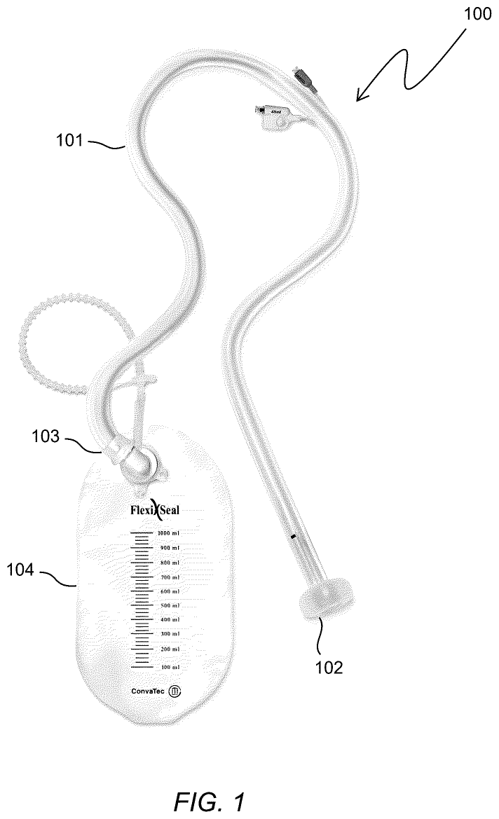

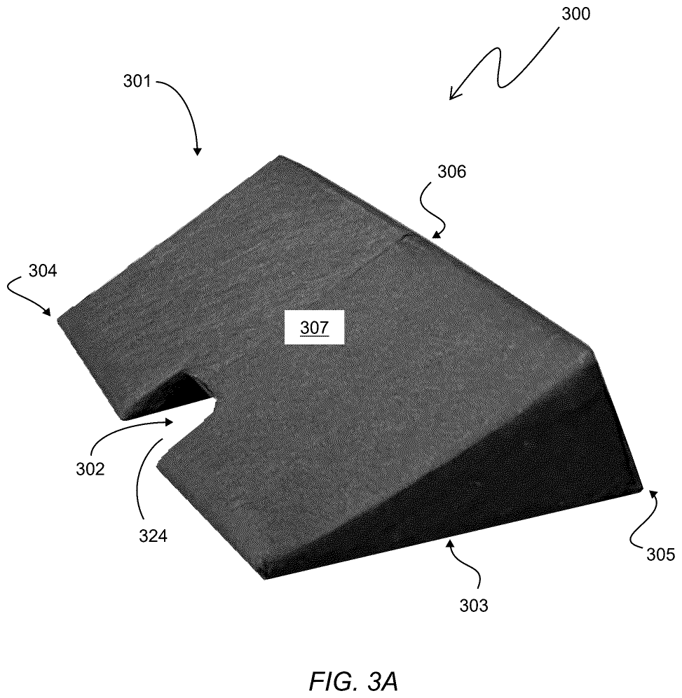

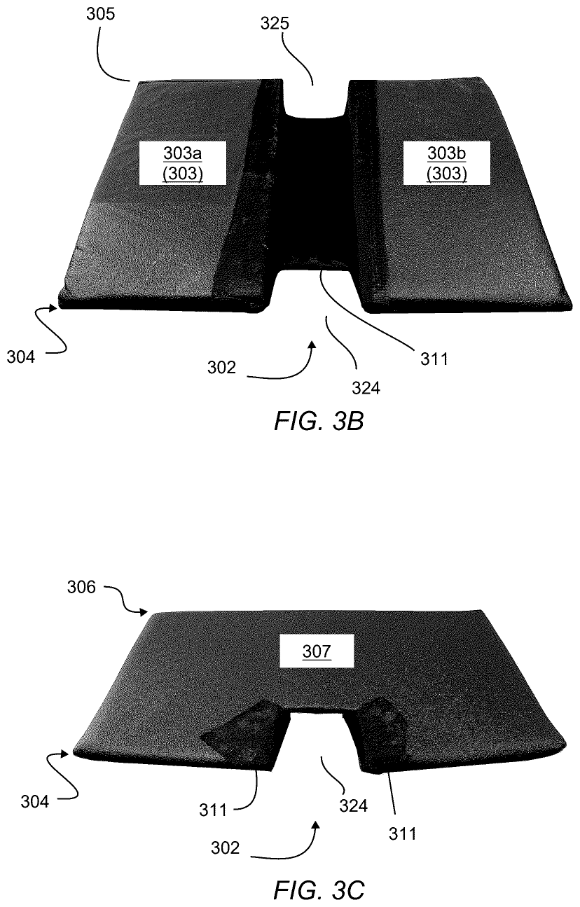

FIGS. 3A, 3B, and 3C are various views of an exemplary positioning device 300. The device 300 comprises a wedge 301 and a channel 302. The wedge 301 comprises an underside 303 configured to rest on a surface such as a bed surface (which may include bedding materials or other coverings). The channel 302 on the underside 303 is configured to accommodate a fecal management catheter (e.g., catheter 101 of FIG. 1) to prevent leaks and improve waste containment.

The wedge 301 comprises a back end 304 and a front end 305. A height of the wedge 301 increases from the back end 304 toward the front end 305. In this exemplary embodiment, there is a single peak 306 where the wedge 301 has the greatest height, and the peak 306 occurs a short distance rearward of the front 305. Accordingly, the increase in wedge height beginning from the back end 304 does not continue all the way to the front end 305 but rather reaches a maximum height at peak 306.

The channel 302 opens at the back end 302 of the wedge 301. The channel 302 also opens at the front end 305. The channel 302 runs from end-to-end on the underside 303 of the wedge 301.

FIG. 3A shows the device 300 oriented according to its state of use, that is, the underside 303 faces down and the topside 307 faces up. FIG. 3B shows the wedge device 300 turned over (i.e., upside down from its orientation in a state of use) for visibility of an entirety of the channel 302. Here it is clearly visible that the channel 302 runs the full distance between the back end 304 and front end 305. The channel 302 has an opening 324 at the back end 304 and an opening 325 at the front end 305. To either side of the channel 302 are underside surfaces 303a and 303b which are parts of the underside 303. The underside surfaces 303a and 303b are configured for contact with the surface onto which the device 300 is placed for use, e.g., on top of a bed.

In FIG. 3A, device 300 is depicted with a uniform exterior material on all exterior surfaces. Device 300 may be used in this condition for some embodiments or, alternatively, be protected with a covering.

In contrast to FIG. 3A, FIGS. 3B and 3C show the underside 303 as comprising a first material for most of surfaces 303a and 303b configured for contact with the underlying surface (e.g., bed) and the channel 302 comprising a second material 311 different from the first material. The second material is non-stick. That is to say, at least one surface of a channel 302 has a lower coefficient of friction than surfaces 303a and 303b of the device. A lower coefficient of friction as with a nonstick surface ensures a catheter, which may be made of silicone or plastic for example, can slide freely within the channel 302 even if in contact with a channel wall. By contrast, a non-stick surface is undesirable for surfaces 303a and 303b. Generally, it is desirable that the device 300 remains in a deliberately set position relative to the bed or other underlying surface. While the nonstick material 311 may wrap partially from the tunnel 302 surfaces onto the neighboring surfaces 303a and 303b to a small extent as depicted by FIG. 3B, it is desirable that at least a majority of the underside 303 which is intended for contact with the surface under the device 300 in a state of use (e.g., a bed) have a relatively high coefficient of friction to prevent the device 300 from slipping or shifting undesirably. Likewise, it is generally desirable for the topside 307 to have a relatively high coefficient of friction (at least relative to the channel 302) so that if a patient is supported by the topside 307, the patient will not accidently slip off the topside 307. As depicted in FIG. 3C, the nonstick material 311 may nevertheless extend from the tunnel 302 partially onto the topside 307. Such an arrangement may be desirable so that a catheter which is routed left or right as it exits the channel 302 does not encounter undue friction from incidental contact which may occur with topside 307.

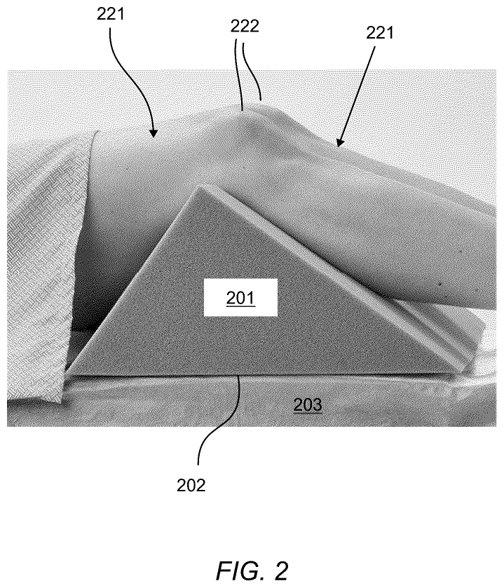

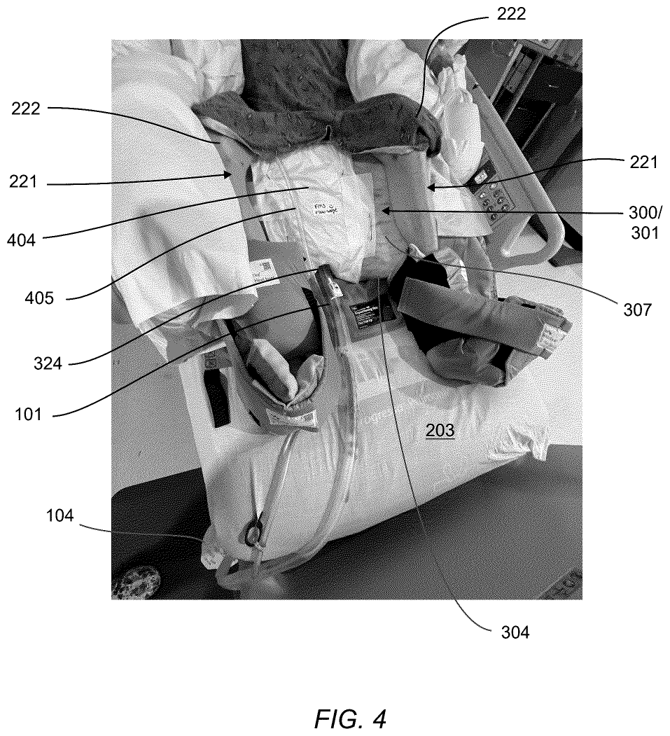

FIG. 4 is an image of positioning device 300 in a state of use. An exemplary method of fecal management is exemplified by FIG. 4. Liquid to semi-liquid stool of the patient is collected through a fecal management catheter 101. The catheter 101 serves as conduit which contains the fecal waste from the patient through to the collection bag/receptacle 104. The fecal management catheter 101 is accommodated in the channel of the positioning wedge 301 to prevent leaks and improve waste containment. FIG. 4 shows the catheter 101 exiting the opening 324 of the channel which is at the underside of the wedge 301. As exemplified by FIG. 4, an exemplary method may further comprise positioning the patient around the fecal management catheter 101 with the positioning wedge 301. As FIG. 4 illustrates, the shape and size of the positioning wedge 301 are configured so that the wedge 301, in particular the topside 307, supports either leg 221 of the patient with the patient knees 222 generally aligned over the peak of the wedge 301. The legs 221 are positioned above and to opposite sides of the catheter 101. In contrast to the conventional wedge 201 of FIG. 2, which would get in the way and risk crushing any catheter which is present, the positioning device 300 in FIG. 4 provides a purposeful and protective channel for routing the fecal management catheter 101 with respect to the patient, the wedge 301, and other objects present for the patient's care. The positioning device 300 accommodates and protects the catheter 101 while simultaneously maintaining a desired alignment of the patient in the bed, e.g. hob 30 degrees, hips aligned with indicators on bed rail.

For non-limiting illustration, FIG. 4 further shows the use of a partial covering 404 to the wedge 301. A urinary catheter 405 separate and apart from fecal management catheter 101 is also visible. A device 300 may have a urinary catheter accommodated in a channel. However, in FIG. 4, the urinary catheter 405 is routed above the positioning device 300.

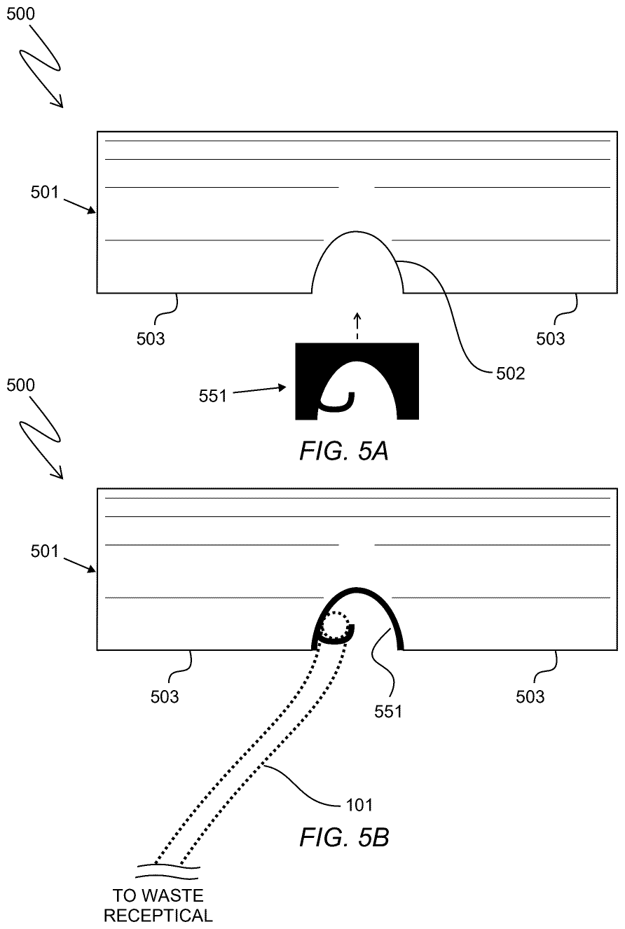

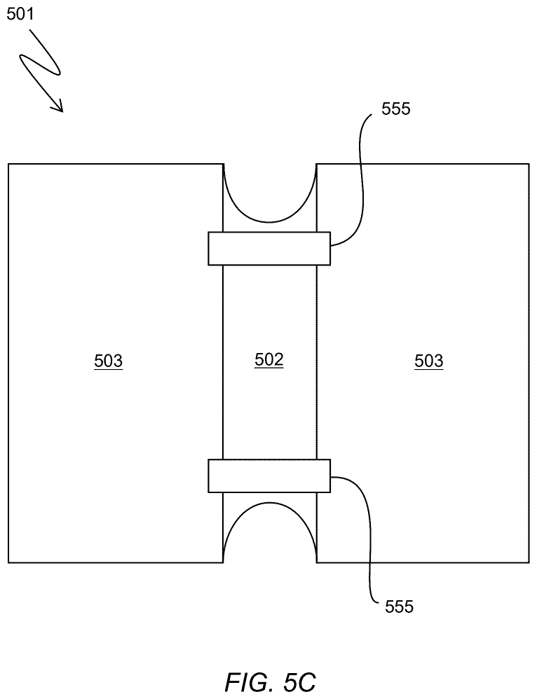

FIGS. 5A, 5B, and 5C show an exemplary positioning device 500 which, alike to positioning device 300, comprises a wedge 501 and a channel 502 on the underside of the wedge configured to accommodate a fecal management catheter 101 to prevent leaks and improve waste containment. The positioning device 400 further comprises at least one retainer 551 configured for restricting the fecal management catheter from exiting the channel from incidental lateral displacement (toward the wedge's left side or right side). The at least one retainer 551 in this exemplary embodiment is an insert which, when inserted into wedge 501, supports the catheter 101 a nonzero distance above the bottom of the channel. The bottom of the channel corresponds with the plane of the underside surfaces 503. Underside surfaces 503 are configured to rest on a bed surface or other support surface. FIG. 5A shows the retainer 551 apart from the wedge 501. FIG. 5B shows the retainer 551 fully inserted and seated in wedge 501.

FIG. 5C is a bottom plan view of the wedge 501. Channel 502 is visible running from end-to-end of the wedge with underside surfaces 503 to either side. At one or more positions along the length of the channel 502 recesses 555 are provided each sized to receive and hold (e.g., by a friction fit) an insert 551.

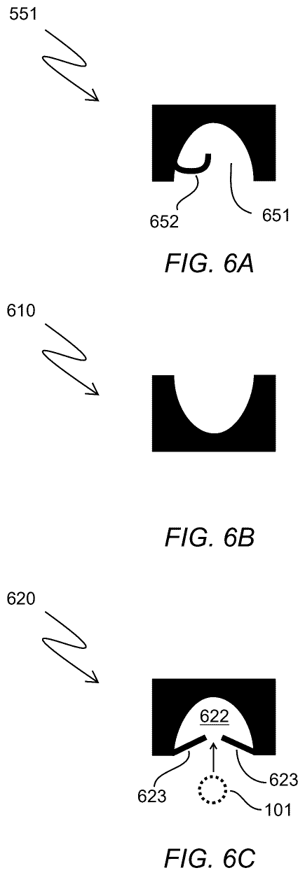

FIG. 6A shows insert 551 alone. The insert 551 comprises a channel 651. A protrusion 652 protrudes from a wall of the channel 651 to create a shelf, ledge, hook, or like structure which is sized and shaped to accommodate a waste management catheter.

FIG. 6B shows an alternative insert 610. The insert 610 is has a U shape. When inserted into a wedge recess, the upward opening of insert 610 and downward opening of the wedge's channels meet to form a fully closed polygon.

FIG. 6C shows a further alternative insert 620. The insert 620 comprises a channel 622. To insert a catheter 101 into the channel 622 along the direction indicated in FIG. 6C, light pressure must be applied to deflect opposing protrusions 623 to permit the catheter 101 to pass. The protrusions 623 elastically then return to their original positions. In their original positions, the space between the ends of the protrusions 623 does not permit the catheter 101 to pass absent a deliberate application of force to the catheter 101 to again deflect the protrusions 623 so as to increase the spacing between the ends of the protrusions 623.

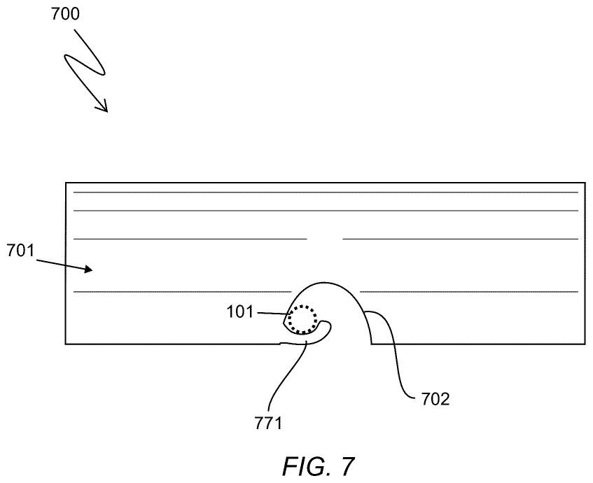

FIG. 7 is a further exemplary positioning device 700 which includes a wedge 701 and a channel 702. The positioning device 700 further comprises at least one retainer 771 configured for restricting a fecal management catheter 101 from exiting the channel 702 from incidental lateral displacement. Here the retainer 771 is a continuous part of the body of the wedge 701. The retainer 771 may be charactered as a notch or protrusion in/from a side of the channel 702.

FIG. 8 is a further exemplary positioning device 800 including a wedge 801 and a channel 802 at an underside of the wedge 801. Notable differences between device 800 and device 300 of FIGS. 3A-3C is that, owing to a larger surface 881 at the back end of the wedge, the opening 824 exists only in the surface 881. By contrast, the opening 324 opens both rearward and upward as is apparent from FIG. 3A, for example. In addition, the peak of the wedge 801 is at the frontmost extent of the wedge 801.

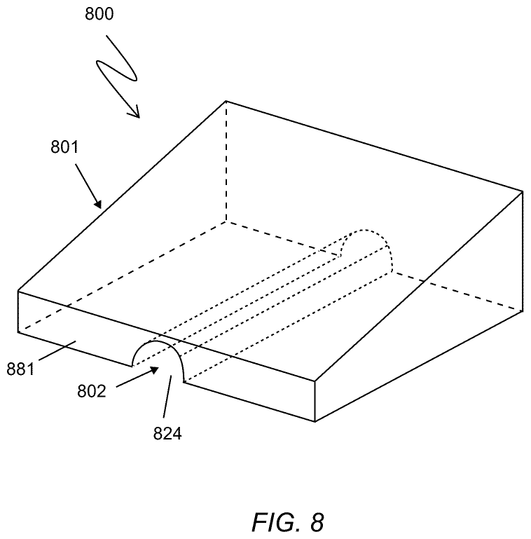

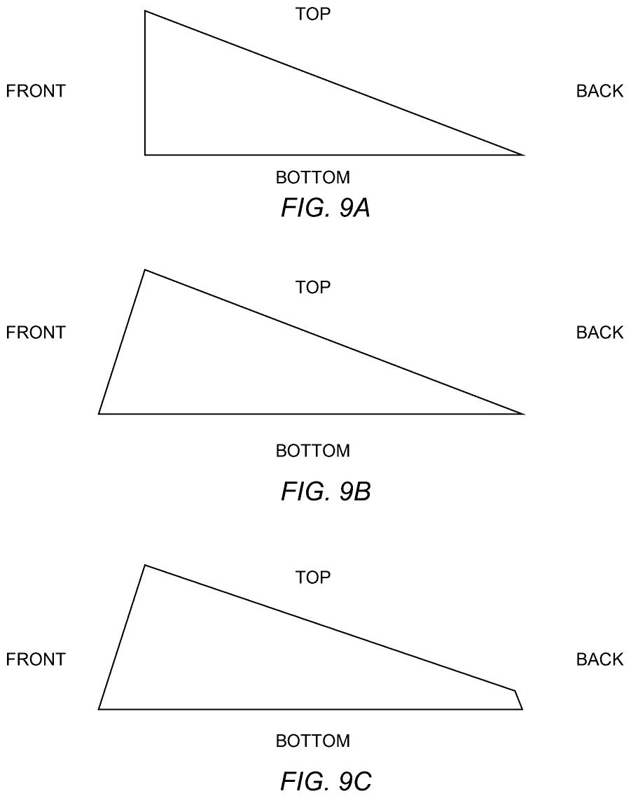

Embodiments may vary as the particular shape of the body of the positioning device. In general, wedge shapes are advantageous and are used for exemplary purposes in many of the drawings of this disclosure. However, bodies of exemplary positioning devices may be shaped in other configurations besides wedge. Furthermore, even for embodiments which include a wedge body, the particular shape of the wedge may vary considerably. As a few non-limiting examples, FIGS. 9A, 9B, and 9C present a few different side profiles of different exemplary wedges.

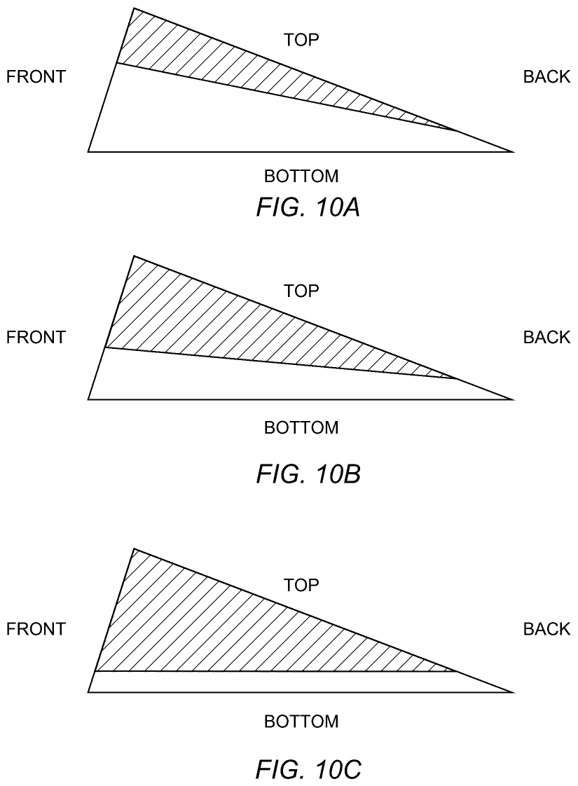

Channels may also vary among embodiments. As non-limiting illustration, FIGS. 10A, 10B, and 10C show cross-sections of exemplary wedges with the cross-section taken along the centerline of each wedge's respective channel. The figures illustrate alternative sizes for the channel at the front end of each wedge. Channels may also vary from one another according to their size at the back end of the wedge. Channels may also vary from one another as to whether the ceiling of each channel is flat or sloped. In any case, the channel is sized and shaped so that an entirety of the catheter may be situated inside the channel for the length of the channel. Because the channel is non-collapsible, the channel prevents external forces, especially those from above, from transferring to the catheter and risking deformation, crushing, or collapse of the catheter.

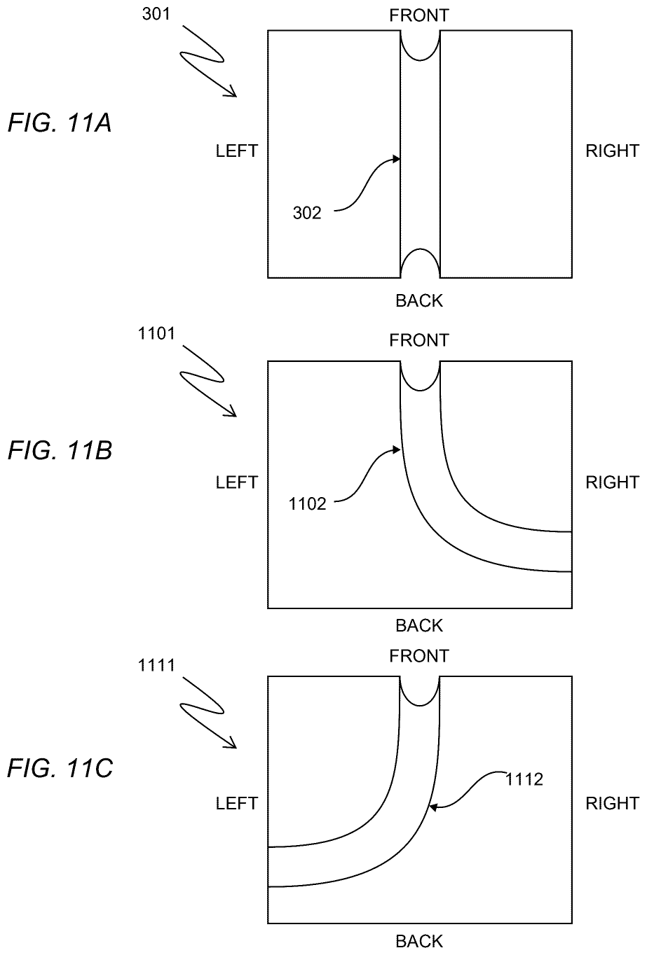

Channels may vary according to which sides of a positioning device they open. FIGS. 11A, 11B, and 11C show a few non-limiting examples of channel configurations according to bottom plan views of various wedges. As exemplified by FIG. 11A, wedge 301 includes a channel which is continuous between front and back sides of the wedge. As one alternative, FIG. 11B shows a wedge 1101 which has a channel 1102 which is continuous between a front side and right side of the wedge. As another alternative, FIG. 11C shows a wedge 1111 which has a channel 1112 which is continuous between a front side and a left side of the wedge.

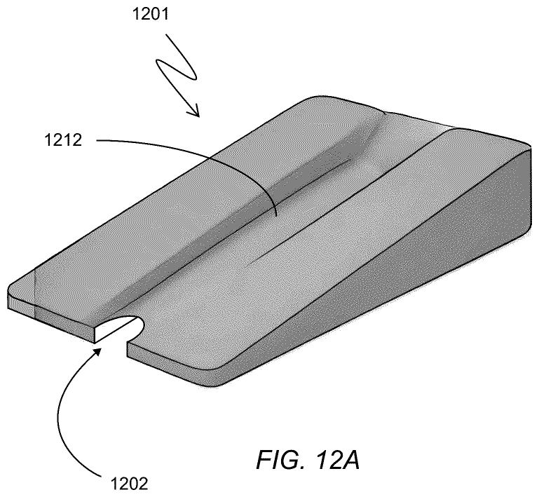

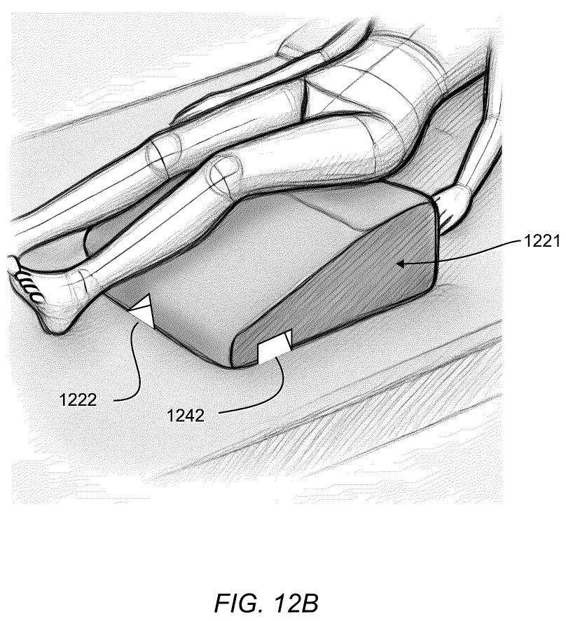

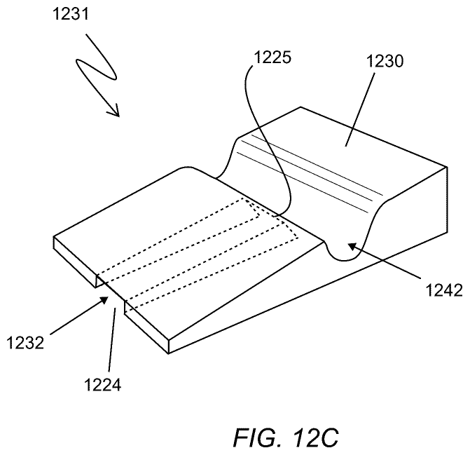

Exemplary positioning devices may be configured with various additional features which are advantageous for particular contexts of use. FIG. 12A, for example, shows an exemplary wedge 1201 which includes a channel 1202 for accommodating a fecal management catheter. The channel 1202 is on an underside of the wedge 1201. The wedge 1201 further comprises a channel 1212 on a topside of the wedge. The channel 1212 is not well-suited for placement of a fecal management catheter but may be included to serve other desirable functions such as, for instance, increased airflow around or beneath a patient.

FIG. 12B depicts a further wedge 1221 which exemplifies further alternative configurations for exemplary positioning devices. The wedge 1221 has a uniquely different shape than some wedges of preceding figures. Two alternative openings 1222 and 1242 for a catheter-accommodating channel are also depicted in alternative sides of the wedge 1221. Channels according to this disclosure may have any of a variety of cross-sectional profiles. An inverted curved U-shape profile is depicted in many of the preceding figures. As further non-limiting alternatives, FIG. 12B shows an inverted V-shape and straight-sided inverted U-shape profile for channels.

FIG. 12C is a further exemplary wedge 1231. The wedge 1231 is distinct for having a fecal management catheter accommodating channel 1232 which does not extend for an entirety of the length of the wedge 1231. The channel 1232 runs between back opening 1224 and frontward facing opening 1225. The opening 1225 is not at the furthermost front side of the wedge 1231. Rather, the channel 1232 ends with opening 1225 in a cross channel 1242 which opens upward. The wedge 1231 is intended for a use case in which a patient is positioned with butt and anus situated in the cross channel 1242. Portion 1230 of the wedge may then support at least the lower back of the patient. Within portion 1230, a channel at the underside of the wedge is not necessary.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

It is to be understood that the terminology and explanations used herein are for the purpose of describing exemplary embodiments only, and are not intended to be limiting, since the scope of the present invention will be limited only by the appended claims.

1. A fecal management system (FMS), comprising
a fecal management catheter configured for collection of liquid to semi-liquid stool; and
a positioning device, comprising
a channel on an underside of the positioning device configured to accommodate the fecal management catheter and allow flow of the liquid to semi-liquid stool at the underside of the positioning device.

2. The FMS of claim 1, wherein the underside comprises a first material for contact with the bed surface and the channel comprises a second material different from the first material, wherein the second material is non-stick.

3. The FMS of claim 1, wherein the positioning device comprises a wedge.

4. The FMS of claim 3, wherein the wedge comprises a back end and a front end, wherein a height of the wedge increases from the back end toward the front end, and wherein the channel opens at the back end of the wedge.

5. The FMS of claim 3, wherein the wedge comprises a back end and a front end, wherein a height of the wedge increases from the back end toward the front end, and wherein the channel opens at both the front end of the wedge and the back end of the wedge.

6. The FMS of claim 1, further comprising at least one retainer configured for restricting the fecal management catheter from exiting the channel by incidental lateral displacement.

7. The FMS of claim 6, wherein the at least one retainer comprises a notch or protrusion in a side of the channel and/or an insert which supports the catheter a nonzero distance above the bottom of the channel.

8. A positioning device for fecal management, comprising
a shape comprising an underside configured to rest on a bed surface; and
a channel on the underside configured to accommodate a fecal management catheter to prevent leaks and improve waste containment.

9. The positioning device of claim 8, wherein the underside comprises a first material for contact with the bed surface and the channel comprises a second material different from the first material, wherein the second material is non-stick.

10. The positioning device of claim 8, wherein the shape is a wedge.

11. The positioning device of claim 10, wherein the wedge comprises a topside which includes a surface directly above the channel.

12. The positioning device of claim 10, wherein the wedge comprises a back end and a front end, wherein a height of the wedge increases from the back end toward the front end, and wherein the channel opens at the back end of the wedge.

13. The positioning device of claim 10, wherein the wedge comprises a back end and a front end, wherein a height of the wedge increases from the back end toward the front end, and wherein the channel opens at the front end of the wedge.

14. The positioning device of claim 10, wherein the wedge comprises a back end and a front end, wherein a height of the wedge increases from the back end toward the front end, and wherein the channel opens at both the front end of the wedge and the back end of the wedge.

15. The positioning device of claim 8, further comprising at least one retainer configured for restricting the fecal management catheter from exiting the channel from incidental lateral displacement.

16. The positioning device of claim 15, wherein the at least one retainer is a notch or protrusion in a side of the channel.

17. The positioning device of claim 15, wherein the at least one retainer is an insert which supports the catheter a nonzero distance above the bottom of the channel.

18. A method of fecal management, comprising
collecting liquid to semi-liquid stool of a patient through a fecal management catheter; and
accommodating the fecal management catheter in a channel of a positioning device to prevent leaks and improve waste containment.

19. The method of claim 18, further comprising positioning the patient around the fecal management catheter with the positioning device.

20. The method of claim 19, further comprising maintaining with the positioning device a desired alignment of the patient in a bed.
